(19) United States
(12) Patent Application Publication
(10) Pub. No.: US 2021/0205317 A1
(43) Pub. Date: Jul. 8, 2021

(54) SYNERGISTIC NEUTRACEUTICAL FORMULATIONS

(71) Applicant: GAMMA DELTA IP, INC., Houston, TX (US)

(72) Inventors: Jason Kelly Allison, Houston, TX (US); Guillermo A. Amtmann, Houston, TX (US); John Burton Steele, Houston, TX (US); Nanda G. Karri, Houston, TX (US)

(21) Appl. No.: 17/210,259

(22) Filed: Mar. 23, 2021

Related U.S. Application Data
(63) Continuation-in-part of application No. 15/621,622, filed on Jun. 13, 2017, now abandoned.
Application No. 15/621,622 is a continuation of application No. 15/369,330, filed on Dec. 5, 2016, now abandoned.
Application No. 15/369,330 is a continuation-in-part of application No. 15/225,391, filed on Aug. 1, 2016, now abandoned.

(51) Int. Cl. A61K 31/522; A61K 9/68; A61K 47/10; A61K 31/137; A61K 31/455; A61K 31/197; A61K 31/4415; A61K 31/4188; A61K 31/519; A61K 31/714; A61K 36/74; A61K 47/44; A61K 47/06; A61K 47/32

(52) U.S. Cl. CPC A61K 31/522; A61K 9/0058; A61K 47/10; A61K 31/137; A61K 31/455; A61K 31/197; A61K 47/32; A61K 31/4188; A61K 31/519; A61K 31/714; A61K 36/74; A61K 47/44; A61K 47/06; A61K 31/4415

(57) ABSTRACT

One or more synergistic nutraceutical chewable formulations providing enhanced thermogenesis leading to enhanced/improved mental concentration and stamina while minimizing adrenaline and dopamine withdrawal. The primary ingredients comprising caffeine, hordenine, and β-phenylethylamine, which are delivered in a chewable form, such as chewing gum, for release and consumption.

### CROSS REFERENCE TO RELATED APPLICATIONS

The present application is a Continuation in Part and claims priority to co-pending U.S. patent application Ser. No. 15/621,622 filed on Jun. 13, 2017, titled “NUTRACEUTICAL CAPSULE AND TABLET FORMULATIONS PROVIDING ENHANCED MENTAL CLARITY, CONCENTRATION AND STAMINA WHILE MINIMIZING ADRENALINE AND DOPAMINE CONCENTRATION PERTUBATIONS ASSOCIATED WITH WITHDRAWAL”, which is a Continuation and claims priority to U.S. patent application Ser. No.: 15/369,330 filed Dec. 5, 2016, titled “NUTRACEUTICAL CAPSULE AND TABLET FORMULATIONS PROVIDING ENHANCED MENTAL CLARITY, CONCENTRATION AND STAMINA WHILE MINIMIZING ADRENALINE AND DOPAMINE CONCENTRATION PERTURBATIONS ASSOCIATED WITH WITHDRAWAL”, which is a Continuation-In-Part and claims priority to U.S. patent application Ser. No. 15/225,391 filed Aug. 1, 2016, titled “Synergistic Nutraceutical Beverage Formulations Providing Enhanced Thermogenesis, Mental Clarity and Stamina While Minimizing Adrenaline and Dopamine Concentration Perturbations Associated With Withdrawal”. The entire contents of these applications are hereby incorporated by reference.

### FIELD

The present disclosure generally relates to nutritional and nutraceutical products, particularly to formulations for oral consumption providing nutritional components that improve both physical and mental aspects of the human condition via thermogenesis delivered in a chewable formulation, such as chewing gum.

More specifically, the chewable formulations in this disclosure are designed to boost energy through thermogenesis enhancing compounds as well as increasing mental concentration and stamina while also (and in some cases) simultaneously reducing or eliminating perturbations existing during adrenaline and dopamine withdrawal.

Perturbations are defined as rapid changes in concentration levels over relatively short time periods. In this case short time periods for human consumption are normally less than 5-6 hours.

### BACKGROUND

A nutraceutical is a food or food product that provides health and medical benefits, including the prevention, treatment, and enhancement of the human condition and in many cases, for the benefit of mammals in general. Most vitamins are made from “food grade” chemicals but a nutraceutical is generally made from pharmaceutical grade chemicals of higher purity and with more predictable and consistent results. Thermogenesis is a term which literally means “the creation of heat”. Often, the term thermogenesis is used as a synonym for the term “energy”. Energy is transformed, converted, transported, and stored within the human body. Human energy comes into the body from what is normally consumed in the form of food and/or beverage. In many cases, beverages contain food-based substituents including vitamins, minerals, and additional supplements which are contained in many of the foods we consume. One unit of the measurement of heat energy involves the use of the terms “joules or kilocalories” and often kilocalories are counted on food and beverage labels in terms of “calories”. Specifically, a calorie is the amount of heat needed to heat one gram of water one degree Celsius. Calories are the unit of measurement describing how much energy is stored in a food or beverage. Water does not contain any calories; however, it is normally found in the food and beverages consumed by humans, and as we consume these substances, energy is either transformed or stored.

As energy is transported through the body's system, some energy leaves the body as fecal energy. Other energy is lost through the urinary tract. Energy not lost through waste by these two means is available for ensuring at least baseline (or basal) metabolic functions. One of the largest energy expenditures in the human body is thermic (heat) energy. Thermic energy differentiates an endoderm (mammal) from an ectoderm (reptile). The endoderm' s basal metabolism is 8 to 10 times higher than that of most ectoderms. Therefore, mammals, and in particular humans, utilize tremendous amounts of the energy that is converted into thermic energy. Energy not used for thermic energy is then available as net energy for the body's cellular reproduction, growth (especially in children), work (muscle movement), and storage. The most common storage form of thermic energy is known as fat.

There are at least three forms of thermogenesis. The first form is work-induced from exercise. It is necessary for human muscles to create or utilize heat in order to work more effectively than when the muscles are cold.

A second form of thermogenesis is known as thermo-regulatory thermogenesis. This form provides for keeping the temperature of the human body regulated. The average body temperature is 98.7 degrees (F). In addition, there are two types of thermo-regulatory thermogenesis: shivering and non-shivering. Shivering helps the body create heat. The skeletal muscles create the shivering. There's a small amount of muscle on each hair that can regulate heat gain and loss.

Non-shivering thermogenesis fits the third classification form, which is also known as diet-induced thermogenesis. Eating a meal or drinking other than water, will produce diet-induced thermogenesis. Normally, humans require more energy to digest food and/or beverages than are being consumed. There are regions within the body that can measure the most critical phenomena regarding the purpose of this disclosure; namely non-shivering or diet-induced thermogenesis.

Diet-induced thermogenesis is very important in animals that hibernate, such as bears, or small animals with a very large surface area in comparison with their body weight. Brown adipose or “brown fat” tissue is where a significant amount of the energy is stored in the body. “Brown fat” is also very prevalent in newborn babies as they exhibit tremendous amounts of non-shivering thermogenesis to regulate their body temperature. As humans age, this system depletes somewhat but remains an important part of survival.

Brown fat (adipose tissue) is located around blood vessels and major organs. When it is actively triggered, it causes the warming of the blood. Warm blood is circulated throughout the body to spread this energy in the form of heat for warming the entire body. The body's thermogenic system triggers the sympathetic nervous system. Under conditions of cold or when larger portions of calories are consumed, the hypothalamus gland registers this energy consumption and then triggers the sympathetic nervous system (also known as an automatic nervous system). The sympathetic nervous system controls many vital functions including heartrate, blood pressure (the circulatory system) and breathing (the respiratory system). Thus, the autonomic (involuntary) nervous system continuously requires and consumes energy.

In the area of the body where the nerves transmit biochemical signals via pathways, one of these neuro “transmitters” is known as norepinephrine. The triggering of the sympathetic nervous system causes the release of norepinephrine from a nerve terminal across the synapse which subsequently binds to receptors to propagate a nerve impulse.

This process increases (in some cases rapidly and dramatically) thermogenesis as well as other activity related functions generated by the nervous system. One way to visualize these somatic (body) functions, is by likening it to raising the body's thermostat. All humans have a basal metabolism rate which can be measured and indicates (in a rather precise manner), the energy required for maintaining vital functions. It is possible, by using formulations described in the present disclosure, to modulate or raise the metabolism rate above the basal rate.

Sweating or perspiration is often associated with one symptom of this increase in thermogenesis. Sweating occurs when more energy is used during thermogenesis of the sympathetic system than the energy used for work related activities. In the case of sweating, less energy is stored, and less energy is converted to fat as opposed to when the body is operating at its basal metabolic rate. In this case, less brown fat is activated, thereby further reducing the metabolism of white fat cells, which are the primary fat storage depository in the human body. It is worth noting that although humans have a consistent number of fat cells, the size can greatly vary. The number of fat cells remains the same, but the size changes.

The brown fat cell is unique in its mitochondrion. Mitochondria are the “energy factories”—oxidative phosphorylation sites—for fueling the body. In the brown fat cell, unique mitochondria help create energy. The brown fat cell is also an energy consumer and it is heat energy that is being consumed. It has been postulated that much of human obesity is less associated with eating habits than it is with brown fat cell deterioration. If these cells deteriorate, energy use and release can be severely compromised. Brown fat activity studies are underway which indicate that post-obese people have a deficiency in their brown fat system.

Another symptom of thermogenesis is a loss of appetite. Exercise also increases the metabolic rate above the basal level giving the feeling of more energy due to the fact that more energy has been transported to the red and white blood cells responsible for (among numerous additional functions) oxygen transport and waste removal.

When an intentionally induced form of thermogenesis is created, by for example, the use of nutritional or nutraceutical supplementation with food or beverages, people feel they have more energy. Often, they are generally able to perform more work-related operations (both physical and mental), which also leads to an increased utilization of fat as the stored calories are converted into released calories. When calories released from the body exceed those consumed, body weight is reduced.

In the past, many of the types of formulations that were designed to provide thermogenesis leading to increased and improved mental stimulation and stamina have also provided deleterious side effects or “symptoms”. The chewable and/or water-soluble formulations of this disclosure have been carefully and precisely formulated to reduce and, in some cases, completely eliminate these deleterious, unwanted side effects or symptoms.

### DETAILED DESCRIPTION OF THE EMBODIMENTS

Before explaining the present disclosure in detail, it is to be understood that the disclosure is not limited to the specifics of particular embodiments as described and that it can be practiced, constructed, or carried out in various ways.

While embodiments of the disclosure have been shown and described, modifications thereof can be made by one skilled in the art without departing from the spirit and teachings of the disclosure. The embodiments described herein are exemplary only and are not intended to be limiting.

Specific structural and functional details disclosed herein are not to be interpreted as limiting, but merely as a basis of the claims and as a representative basis for teaching persons having ordinary skill in the art to variously employ the present embodiments. Many variations and modifications of embodiments disclosed herein are possible and are within the scope of the present disclosure.

Where numerical ranges or limitations are expressly stated, such express ranges or limitations should be understood to include iterative ranges or limitations of like magnitude falling within the expressly stated ranges or limitations.

The use of the word “a” or “an” when used in conjunction with the term “comprising” in the claims and/or the specification may mean “one,” but it is also consistent with the meaning of “one or more,” “at least one,” and “one or more than one.”

The word “about”, when referring to values, means plus or minus 5% of the stated number.

The use of the term “optionally” with respect to any element of a claim is intended to mean that the subject element is required, or alternatively, is not required. Both alternatives are intended to be within the scope of the claim. Use of broader terms such as comprises, includes, having, etc. should be understood to provide support for narrower terms such as consisting of, consisting essentially of, comprised substantially of, and the like.

When methods are disclosed or discussed, the order of the steps is not intended to be limiting, but merely exemplary unless otherwise stated.

Accordingly, the scope of protection is not limited by the description herein, but is only limited by the claims which follow, encompassing all equivalents of the subject matter of the claims. Each and every claim is hereby incorporated into the specification as an embodiment of the present disclosure. Thus, the claims are a further description and are an addition to the embodiments of the present disclosure.

The inclusion or discussion of a reference is not an admission that it is prior art to the present disclosure, especially any reference that may have a publication date after the priority date of this application. The disclosures of all patents, patent applications, and publications cited herein are hereby incorporated by reference, to the extent they provide background knowledge; or exemplary, procedural or other details supplementary to those set forth herein.

Generally, the present disclosure describes synergistic nutraceutical chewable formulations providing enhanced thermogenesis leading to enhanced/improved mental concentration and stamina while minimizing adrenaline and dopamine withdrawal. In most cases these synergistic effects are simultaneous within the consumer's body and are primarily due to the ability to slow the metabolic rate of caffeine and (β-phenylethylamine withdrawal when combined with the MAO-B inhibitor, hordenine HCL. It is also clear that combining caffeine, β-PEA, and hordenine (likely) results in a synergistic residual reduction in the withdrawal symptoms associated with catecholamine withdrawal following consumption of the beverage formulation(s) due to the increased half-life of β-PEA. These symptoms are more fully described below.

In order to extend physical and mental stamina via thermogenesis and (in some cases) simultaneously reduce unwanted withdrawal symptoms, the present disclosure describes a combination of caffeine, hordenine, and β-phenylethylamine (β-PEA) in specific doses together with a B-vitamin “complex” and water with sugars or sugar substitutes (inactives).

This chewable and/or water-soluble formulation provides not only an increase in thermogenesis associated with a feeling of increased energy due to increased physical metabolic activity, but also stimulates and enhances brain function related to mood and focus. The word “complex” in this case refers to the use of one or more of the B vitamins as listed below but should not be construed as being a specific combination of B vitamins that has been complexed in any particular combination prior to providing the beverage of the present disclosure.

The vitamin B portion is also a composition that comprises, in any combination, some of the water-soluble B vitamins, specifically; B3 (Niacinamide) and/ or B6 (pyroxidine hydrochloride), B9, folate (folic acid), B12 (cyanocobalamin), B7 (biotin) and B5 (pantothenic acid). Vitamin B3 can be present in amounts ranging from about 0.002 g to 0.20 g, vitamin B5 as pantothenic acid is present in the ranges from 0.000001 g to 0.100 g, vitamin B6 can be present in amounts ranging from 0.0002 g to 0.20 g, vitamin B7, in the form of Biotin can be present in the amounts ranging from 0.00001 g to 0.01 g, and vitamin B12 can be present in amounts ranging from around 0.000001 g to 0.50 g. Furthermore, vitamin B12 can be dosed as cyanocobalamin (a form that the body readily converts to the active forms methylcobalamin and 5-deoxyadenosylcobalamin). Cyanocobolamin can be converted into methylcobolamin and adenosylcobolamin which can be present in amounts ranging from around 0.000001 g to 0.50 g.

This product normally would provide “active” ingredients in chewable form or dispersed in water. Here the term “active” or “actives” is meant to convey the meaning of ingredients that are known to provide active stimulation of thermogenesis and/or brain activity when ingested by humans. Alternatively, the term “inactive” or “inactives” refer to, for example, a simple carrier or binding agent, such as water or primarily inert fillers such as calcium, magnesium, or sodium salts, thickening agents, emulsifying agents, and buffering agents.

In embodiments, these formulations utilize only components and compounds extracted or derived from earth grown or naturally occurring earth-available substances.

In other embodiments, the composition will optionally contain calcium and magnesium salts. Calcium and magnesium salts can be present in amounts ranging from about 0.001 g to 0.50 g,

More specifically, the present disclosure describes one or more nutraceutical chewable and water-soluble formulations providing enhanced thermogenesis, mental concentration, and stamina to mammals, comprising at least three distinct synergistic components including caffeine, β-phenylethylamine, and hordenine, wherein the formulations reduce or eliminate undesirable side effects during or after mammalian ingestion of the formulations.

Hordenine used is typically hordenine HCl, but not limited thereto. The formulations can comprise β-phenylethylamine that is either β-phenylethylamine citrate or β-phenylethylamine HCl or a combination of both.

The synergistic components act to increase catecholamine and indoleamine concentrations and the synergism is accomplished as the catecholamine and indoleamine concentrations cross a blood-brain barrier after consumption, thereby reducing or eliminating acute symptoms associated with rapid decline of the catecholamine and indoleamine concentrations in the blood.

These synergistic components undergo a metabolic breakdown resulting in a controlled decrease in the catecholamine and indoleamine concentrations such that the concentrations revert to a pre-existing basal level associated with the consumer of the beverage formulation.

These synergistic components provide synergy in that β-phenylethylamine decreases serotonin reuptake, hordenine prevents metabolic breakdown of β-phenylethylamine and maintains serotonin levels, while caffeine decreases serotonin levels.

The synergistic component, β-phenylethylamine corrects for reduction of serotonin levels that occur following prolonged consumption of caffeine.

The distinct synergistic components are further combined with one or more B vitamins such that these B vitamins provide additional cognitive enhancing attributes to mental clarity, concentration and stamina. More specifically, the three distinct synergistic components are combined with a B vitamin complex, such that the vitamin B complex includes B3, B5, B6, B7, B9, and B12 mixed together in any combination and at any concentration that will further reduce or eliminate side effects associated with any of the three distinct components and the vitamin B complex during or after human ingestion.

In embodiments, the three distinct synergistic components are combined with one or more sweeteners including sugars and/or sugar substitutes.

In embodiments, the formulations can include an addition of flavors, such as natural flavors and/or organic flavors.

In embodiments, the caffeine used for these beverages is a green coffee bean extract.

In embodiments, the water can be distilled water, deionized water, buffered water, filtered water or any combination thereof. In this case, the buffered water can be pH adjusted with buffering agents to either increase or decrease pH of the distilled, deionized, and/or filtered water.

In embodiments, the formulations can include sugars selected from the group consisting of sucrose, crystalline fructose, high fructose syrups, mannose, dextrose, monk fruit sugar, maple syrup, honey, or combinations thereof.

The sugar substitutes can be selected from: stevia, saccharin, aspartame and sucralose. In the case of stevia, it can be an earth grown derived form of stevia, such as from the stevia leaf.

In embodiments, these formulations can also contain pharmaceutical grade salts selected from the group consisting of calcium salts, magnesium salts, and sodium salts further enhancing the effects associated with these formulations and combinations thereof. Acceptable salts enhance flavor and efficacy of these formulations specific to improving mental clarity, concentration, and both physical and mental stamina.

In a separate embodiment, one or more nutraceutical formulations providing enhanced thermogenesis, mental concentration, and stamina, comprising at least three distinct synergistic components including caffeine, β-phenylethylamine, and hordenine, provide a boost of energy-generating catecholamines from the caffeine including dopamine and norepinephrine which produces amphetamine stimulation and performance enhancement.

In this case, the β-PEA further promotes an efflux of catecholamines which blocks a re-uptake and simultaneously decreases reuptake of serotonin. This decrease may or may not be simultaneous depending on several factors including the consumer's metabolism, concentration of the synergistic components consumed, and the activity before, during and after the consumer has consumed these beverage formulations.

In addition, the hordenine stabilizes the β-PEA in that the β-PEA remains in its pre-consumed form for an extended length of time, normally several hours. Without the addition of hordenine the β-PEA would destabilize and become metabolically consumed much more rapidly.

The β-PEA is a stronger NDRI (Norepinephrine-Dopamine Reuptake Inhibitor) and is a weaker-acting SSRI (Selective Serotonin Reuptake Inhibitor) in terms of providing this type of activity across the blood-brain barrier when consumed by the consumer.

The synergistic action of β-PEA with hordenine and caffeine provide prolonged mental and physical benefits and diminish or eliminate unwanted withdrawal symptoms after consumption by reduction of adrenaline and dopamine perturbations.

In developing these formulations, it has been determined that there are several methods associated with making one or more nutraceutical formulations for increasing thermogenesis.

One such method comprises providing at least three distinct synergistic components including caffeine, β-phenylethylamine, and hordenine, and water, such that the components are provided in powder form to be dissolved in water.

Additionally, components including sugar, sugar substitutes, B vitamins, colorants and flavorings can be added to the formulations thereby achieving final formulation compositions for consumption.

These final formulation compositions are used to improve functional conditions in mammals associated with mental clarity, concentration, and stamina.

In embodiments, the components are provided in chewable form, such as in a chewing gum for release when chewed.

Consumption of these nutraceutical formulations includes an administration route selected from the group consisting of oral buccal, sublingual, or combinations thereof.

Most specifically, this disclosure provides for one or more compositions that cause an increase in thermogenesis in mammals, comprising at least caffeine, hordenine and β-phenylethylamine and optionally the addition of B vitamins. The caffeine is often sourced from green coffee bean extracts and normally comprises from 0.0001 g to 0.30 g of caffeine from 0.00001 g to 0.1 g of hordenine sourced from barley and from 0.00001 g to 1.0 g β-phenylethylamine sourced from cocoa beans.

The composition can be delivered in chewable form, such as in a chewing gum. In embodiments, the gum can comprise a gum base, a glycerine, and a plasticizer. The gum can also have a sweetener, a flavor, and a coating. Various fillers can be used to achieve desired texture, weight, and other physical characteristics of the gum.

The gum base is a non-digestible masticatory, or chewable, delivery mechanism for the active ingredients of the present disclosure, i.e., at least caffeine, hordenine, and β-phenylethylamine. In embodiments, the gum base can comprise natural latices of vegetable origin or synthetic polymer latices.

Natural latices of vegetable origin can comprise components such as chicle, beeswax, and/or latex.

Synthetic polymer latices can comprise paraffin, butadiene-styrene, vinyl acetate, and/or polyethylene. Various usable polymers are known to persons having ordinary skill in the art.

The chewable composition can further comprise a glycerine, a plasticizer, a sweetener, a flavor, a coating, and/or filler materials.

It is not the intent of this disclosure to claim a chewing gum, the creation of which is well known to persons having ordinary skill in the art. This disclosure specifically deals with the delivery of the above nutraceutical formulation via a chewable means.

An exemplary embodiment comprises from about 0.0001 g to about 0.30 g of caffeine, from about 0.00001 g to about 0.1 g of hordenine, from about 0.00001 g to about 1.0 g β-phenylethylamine, a gum base from about 25% to about 35%, a glycerine from about 2% to about 10%, a plasticizer from about 1% to about 2%, a sweetener from about 0.5% to about 1%, a flavor from about 1% to about 5%, and a coating as needed.

While the ranges of caffeine, hordenine, and β-phenylethylamine must be maintained, persons having ordinary skill in the art can adjust the composition of the gum delivery mechanism as desired.

While the present disclosure emphasizes the presented embodiments, it should be understood that within the scope of the appended claims, the disclosure might be embodied other than as specifically enabled herein.

What is claimed is:

1. A nutraceutical formulation in form providing enhanced thermogenesis, mental concentration, and stamina to mammals, comprising:
a. a gum base;
b. a glycerine;
c. a plasticizer;
d. a caffeine with a concentration of 0.0001 grams to 0.3 grams;
e. a β-phenylethylamine with a concentration of 0.00001 grams to 1 gram; and
f. a hordenine with a concentration of 0.00001 grams to 0.1 grams; and
wherein the formulation contains only ingredients capable of crossing the blood-brain barrier.

2. The formulation of claim 1, further comprising a sweetener, a flavor, or a coating.

3. The formulation of claim 1, wherein the hordenine is provided as a salt comprising hordenine.

4. The formulation of claim 1, wherein the β-phenylethylamine is provided as a salt comprising β-phenylethylamine.

5. The formulation of claim 1, further comprising at least one B vitamin.

6. The formulation of claim 1, further comprising a B vitamin complex, the B vitamin complex including B3, B5, B6, B7, B9, and B12 mixed together in any combination.

7. The formulation of claim 1, wherein the caffeine is a green coffee bean extract.

8. The formulation of claim 1, wherein the gum base comprises about 25% to about 35% of the formulation.

9. The formulation of claim 1, wherein the gum base comprises natural latices of vegetable origin or synthetic polymer latices.

10. The formulation of claim 1, wherein the glycerine comprises about 2% to about 10% of the formulation.

11. The formulation of claim 1, wherein the plasticizer comprises about 1% to about 2% of the formulation.

12. The formulation of claim 2, wherein the sweetener comprises about 0.5% to about 1% of the formulation.

13. The formulation of claim 2, wherein the flavor comprises about 1% to about 5% of the formulation.

14. The formulation of claim 9, wherein the natural latices comprise chicle, beeswax, or latex.

15. The formulation of claim 9, wherein the synthetic polymer latices comprise paraffin, butadiene-styrene, vinyl acetate, or polyethylene.
